(19) United States
(12) Patent Application Publication
(10) Pub. No.: US 2018/0256496 A1
(43) Pub. Date: Sep. 13, 2018

(54) CONTROLLED-RELEASE FORMULATIONS

(71) Applicant: Camurus AB, Lund (SE)

(72) Inventors: Fredrik Tiberg, Lund (SE); Markus Johnsson, Lund (SE); Justas Barauskas, Lund (SE)

(73) Assignees: Camurus AB, Lund (SE); Camurus AB, Lund (SE)

(21) Appl. No.: 15/761,053

(22) PCT Filed: Sep. 16, 2016
(86) PCT No.: PCT/EP16/72059

§ 371 Date: Mar. 16, 2018

(30) Foreign Application Priority Data
Sep. 18, 2015 (GB) 1516554.1

(51) Int. Cl. A61K 9/00; A61K 31/439; A61K 47/10; A61K 47/14; A61K 47/24; A61P 1/02

(52) U.S. Cl. CPC A61K 9/0021; A61K 31/439; A61K 47/10; A61K 31/485; A61K 47/24; A61P 1/02; A61K 2300/00; A61K 47/14

(57) ABSTRACT

The present invention relates to an injectable pre-formulation comprising a low viscosity mixture of: a) at least one neutral diacyl lipid and/or at least one tocopherol; b) at least one phospholipid; c) at least one oxygen containing organic solvent; d) at least one 5HT₃ antagonist; wherein the pre-formulation forms, or is capable of forming, at least one liquid crystalline phase structure upon contact with an aqueous fluid. Such compositions may additionally comprise polar solvents and/or further active agents such as opioid agonists and/or antagonists. Methods of treatment, particularly for management of nausea and vomiting such as for post-operative nausea and vomiting and/or therapy induced nausea and vomiting are provided, as well as corresponding uses of the compositions. Administration devices comprising the formulations and kits comprising the devices are also provided.

### FIELD

The present invention relates to formulation precursors (pre-formulations) comprising lipids that upon exposure to water or aqueous media, such as body fluids, spontaneously undergo at least one phase transition, thereby forming a controlled release matrix. In particular, the present invention is concerned with pre-formulations comprising at least one 5HT3 antagonist.

### BACKGROUND

Many bioactive agents including pharmaceuticals, nutrients, vitamins and so forth have a “functional window”. That is to say that there is a range of concentrations over which these agents can be observed to provide some biological effect. Where the concentration in the appropriate part of the body (e.g. locally or as demonstrated by serum concentration) falls below a certain level, no beneficial effect can be attributed to the agent. Similarly, there is generally an upper concentration level above which no further benefit is derived by increasing the concentration. In some cases increasing the concentration above a particular level, results in undesirable or even dangerous effects.

Some bioactive agents have a long biological half-life and/or a wide functional window and thus may be administered occasionally, maintaining a functional biological concentration over a substantial period of time (e.g. 6 hours to several days). In other cases the rate of clearance is high and/or the functional window is narrow and thus to maintain a biological concentration within this window regular (or even continuous) doses of a small amount are required. This can be particularly difficult where non-oral routes of administration (e.g. parenteral administration) are desirable or necessary. Furthermore, in some circumstances, such as in the fitting of implants (e.g. joint replacements or oral implants) the area of desired action may not remain accessible for repeated administration. Similarly, patient compliance may limit how regularly and/or how frequently administration can be made. In such cases a single administration must provide active agent at a therapeutic level over an extended period, and in some cases over the whole period during which activity is needed.

Various methods have been used and proposed for the sustained release of biologically active agents. Such methods include slow-release, orally administered compositions, such as coated tablets, formulations designed for gradual absorption, such as transdermal patches, and slow-release implants such as “sticks” implanted under the skin.

One method by which the gradual release of a bio active agent has been proposed is a so-called “depot” injection. In this method, a bioactive agent is formulated with carriers providing a gradual release of active agent over a period of a number of hours or days. These are often based upon a degrading matrix which gradually disperses in the body to release the active agent.

A controlled-release product, especially in ready-made-up form, which is administrable by simple injection offers a number of potential advantages. Such a product requires only a minimum imposition on the time of a health worker since the administration is infrequent (possibly occurring only once per event, such as chemotherapy dose or surgical operation). Ready-to-use products further do not require lengthy preparation, saving time and reducing the possibility of error.

The most common of the established methods of depot injection relies upon a polymeric depot system. This is typically a biodegradable polymer such poly (lactic acid) (PLA) and/or poly (lactic-co-glycolic acid) (PLGA) and may be in the form of a solution in an organic solvent, a pre-polymer mixed with an initiator, encapsulated polymer particles or polymer microspheres. The polymer or polymer particles entrap the active agent and are gradually degraded releasing the agent by slow diffusion and/or as the matrix is absorbed. Examples of such systems include those described in U.S. Pat. No. 4,938,763, U.S. Pat. No. 5,480,656 and U.S. Pat. No. 6,113,943 and can result in delivery of active agents over a period of up to several months.

One alternative to the more established, polymer based, depot systems was proposed in U.S. Pat. No. 5,807,573. This proposes a lipid based system of a diacylglycerol, a phospolipid and optionally water, glycerol, ethylene glycol or propylene glycol to provide an administration system in the reversed micellar “L₂” phase or a cubic liquid crystalline phase. Since this depot system is formed from physiologically well tolerated diacyl glycerols and phospholipids, and does not produce the lactic acid or glycolic acid degradation products of the polymeric systems, there is less tendency for this system to produce inflammation at the injection site. The liquid crystalline phases are, however, of high viscosity and the L₂ phase may also be too viscous for ease of application.

The use of non-lamellar phase structures (such as liquid crystalline phases) in the delivery of bioactive agents is now relatively well established. Such structures form when an amphiphilic compound is exposed to a solvent because the amphiphile has both polar and apolar groups which cluster to form polar and apolar regions. These regions can effectively solubilise both polar and apolar compounds. In addition, many of the structures formed by amphiphiles in polar and/or apolar solvents have a very considerable area of polar/apolar boundary at which other amphiphilic compounds can be adsorbed and stabilised. Amphiphiles can also be formulated to protect active agents, to at least some extent, from aggressive biological environments, including enzymes, and thereby provide advantageous control over active agent stability and release.

The formation of non-lamellar regions in the amphiphile/water, amphiphile/oil and amphiphile/oil/water phase diagrams is a well known phenomenon. Such phases include liquid crystalline phases such as the cubic P, cubic D, cubic G and hexagonal phases, which are fluid at the molecular level but show significant long-range order, and the L₃ phase which comprises a multiply interconnected bi-continuous network of bilayer sheets which are non-lamellar but lack the long-range order of the liquid crystalline phases. Depending upon the curvature of the amphiphile sheets, these phases may be described as normal (mean curvature towards the apolar region) or reversed (mean curvature towards the polar region).

The non-lamellar liquid crystalline and L₃ phases are thermodynamically stable systems. That is to say, they are not simply a meta-stable state that will separate and/or reform into layers, lamellar phases or the like, but are the stable thermodynamic form of the lipid/solvent mixture.

A class of active agents having particular potential as depot or slow-release formulations are 5-HT3 anti-emetics. The term “anti-emetic” as used herein encompasses any drug which is effective at treating and/or preventing nausea and/or vomiting. Anti-emetics may be administered to treat the primary illness, such as in the case of nausea, vomiting, IBS, gastroenteritis and motion sickness, for instance.

Alternatively, anti-emetics may also be used for prophylaxis against side-effects of a course of treatment. In particular, nausea and vomiting are common side effects of cancer treatment, especially associated with chemotherapy, radiotherapy and endoradionulclide therapy. The term “chemically induced nausea and vomiting” (CINV) is used in this context to refer to nausea and vomiting resulting from moderately emetogenic chemotherapy (MEC) or highly emetogenic chemotherapy (HEC). The term “treatment induced nausea and vomiting” (TINV) is used herein to indicate nausea and/or vomiting resulting as an undesirable side-effect of any course of therapeutic or prophylactic treatment such as any of those mentioned herein, including chemotherapy, radiotherapy, endoradionulclide therapy, surgery and/or anaesthesia. Such treatments may be for any purpose including cancer therapy.

Apart from the patient discomfort associated with nausea and vomiting, particularly associated with MEC and HEC, these side effects may cause serious complications, e.g. dehydration. Elderly patients are particularly susceptible to complications resulting from these side effects. Nausea and vomiting are often severe enough that the patient postpones or even refuses treatment. Where a treatment or drug causes nausea and vomiting as side effects, antiemetic agents are often prescribed alongside.

It is known that CINV may be reduced by the administration of 5-HT3 receptor antagonists. Examples of 5-HT3 receptor antagonists approved for use include dolasetron (Anzemet), ondansetron (Zofran), palonosetron (Aloxi) and granisetron (Kytril). Existing products are typically administered intravenously or orally rather than as a slow-release “depot”.

However, one “depot” product being developed to treat the effects of CINV is SUSTOL® by Heron Therapeutics. This product contains the 5-HT3 receptor antagonist granisetron in Biochronomer® matrix, which is a polymer based matrix based around poly(ortho esters), as described for instance in US2014/323517 A1 and US2014/296282 A1.

There are, however, drawbacks to using a polymeric matrix. For instance, polymeric matrices tend to have high viscosities, and therefore are often requiring high organic solvent content to enable injection and may be painful to inject. Additionally, the breakdown products may cause discomfort to the patient, such as in the case of the well-known PLGA system. Polymeric depot systems also tend to exhibit a “burst” profile, i.e. a substantial part of the active agent is released from the matrix in a short period of time after administration, before a stable rate of release is reached. This naturally means that great care regarding dosing is required to avoid the “peak” concentration reaching undesirable levels. It is also not always possible to prepare a long-duration depot product using a polymeric matrix, for instance where two or more therapeutic agents having different physical characteristics are required.

The present inventors have now established that by providing a lipid-based controlled release matrix comprising a 5HT3 receptor antagonist, a slow-release “depot” antiemetic product can be formulated which addresses various deficiencies of existing antiemetic products. In particular, the precursor formulation which forms the depot product on administration has the benefit of being easy to administer, and the depot product releases the antiemetic gradually allowing for the possibility of less frequent dosing. Furthermore, components of particular interest in the present invention are all known to be biotolerable and are known to meet regulatory standards. They are furthermore effective in solubilising actives with a wide range of physical characteristics.

Precursor formulations (“pre-formulations”) of the present invention are particularly suitable for use in the treatment of emesis, nausea, vomiting, chemotherapy and/or radiotherapy and/or endoradionuclide therapy induced nausea and vomiting, post-operative nausea and vomiting, delayed nausea and vomiting optionally accompanied by, pain, post-operative pain and/or extended post-operative pain. This applies particularly in patients undergoing chemotherapy (including HEC and MEC), or having cancer, motion sickness, IBS, gastroenteritis and/or related conditions. Pre-formulations of the invention may also be used to treat opioid dependence and to address issues of nausea and vomiting in opioid dependence treatment.

### SUMMARY OF THE INVENTION

Viewed from a first aspect, the invention provides a pre-formulation comprising a low viscosity mixture of:

a) at least one neutral lipid (e.g. a diacyl lipid such as diacyl glycerol);

b) at least one phospholipid;

c) at least one oxygen containing organic solvent; and

d) at least one 5HT₃ antagonist.

Typically the pre-formulation forms, or is capable of forming, at least one liquid crystalline phase structure upon contact with an aqueous fluid.

In all embodiments the pre-formulation is preferably injectable, by which is intended that the pre-formulations will have properties suitable for injection. Such properties may include sterility (e.g. by sterile filtration), tonicity, viscosity and other factors rendering them suitable for injection.

In all aspects of the invention, salts of 5HT₃ antagonists are preferred. These will be biologically tolerable salts including halides, such as chloride or bromide. A particularly preferred 5HT₃ antagonist is granisetron or a biologically acceptable salt thereof, especially granisetron chloride.

In one embodiment applicable to all aspects of the invention, the pre-formulation may further comprise a polar solvent component e).

In another embodiment applicable to all aspects of the invention, the pre-formulation may further comprise an opioid agonist and/or antagonist—component f). If present, it is particularly preferred that this component comprises buprenorphine or a biologically acceptable salt thereof, especially buprenorphine chloride.

All opioid agonists and/or antagonists may be in the form of their free base and/or in the form of a salt. Suitable salts will be pharmaceutically tolerable and may include halide salts such as chloride or bromide. In one embodiment applicable to all aspects of the invention, the opioid agonist(s) and/or antagonist(s) may be in the form of the free base.

In certain embodiment applicable to all aspects of the invention, the pre-formulation may comprise a 5HT3 antagonist, and at least one opioid agonist, partial agonist and/or antagonist comprising naloxone. In certain embodiments, pre-formulations will comprise a 5HT3 antagonist and at least one opioid agonist, partial agonist and/or antagonist comprising buprenorphine and/or naloxone. Particularly preferred for certain embodiments are pre-formulations comprising granisetron, buprenorphine and naloxone.

Viewed from a second aspect, the invention provides a method for the treatment of a human or non-human mammalian subject in need thereof with a 5HT3 antagonist, said method comprising administering to said subject a pre-formulation comprising a low-viscosity mixture of;

a) at least one neutral lipid (e.g. a diacyl lipid such as a diacyl glycerol);

b) at least one phospholipid;

c) at least one oxygen containing organic solvent; and

d) at least one 5HT₃ antagonist.

Pre-formulations of the invention are particularly suited to the treatment, prevention or partial prevention of at least one condition selected from emesis, nausea, vomiting, chemotherapy and/or radiotherapy and/or endoradionuclide therapy induced nausea and/or vomiting, post-operative and/or extended post-operative nausea and vomiting, pain, post-operative and/or extended post-operative pain, delayed nausea and vomiting in patients undergoing chemotherapy including HEC and MEC, cancer, opioid dependence, motion sickness, IBS, gastroenteritis and/or related conditions.

In one embodiment, the method of treatment involves a single administration every 1 to 28 days. In another embodiment, said administration may be every 1 to 21 days, such as every 1 to 14 days or 1 to 7 days, particularly every 3 to 7 days.

Viewed from a third aspect, the invention relates to the use of;

a) at least one neutral lipid (e.g. a diacyl lipid such as a diacyl glycerol);

b) at least one phospholipid;

c) at least one oxygen containing organic solvent; and

d) at least one 5HT₃ antagonist;

in the manufacture of a low viscosity pre-formulation medicament for use in the in vivo formation of a depot for treatment of at least one condition selected from emesis, nausea, vomiting, chemotherapy and/or radiotherapy and/or endoradionuclide therapy induced nausea and vomiting, post-operative and extended post-operative nausea and vomiting, pain, post-operative and/or extended post-operative pain, opioid dependence, motion sickness, IBS, gastroenteritis and/or related conditions.

Viewed from a fourth aspect, the invention provides a pre-formulation comprising a low viscosity mixture of:

a) at least one neutral lipid (e.g. a diacyl lipid such as a diacyl glycerol);

b) at least one phospholipid;

c) at least one oxygen containing organic solvent; and

d) at least one 5HT₃ antagonist;

for use in the treatment of at least one condition selected from emesis, nausea, vomiting, chemotherapy and/or radiotherapy and/or endoradionuclide therapy induced nausea and vomiting, post-operative nausea and vomiting, pain, post-operative pain, extended post-operative pain, opioid dependence, motion sickness, IBS, gastroenteritis and/or related conditions.

Typically the pre-formulation forms, or is capable of forming, at least one liquid crystalline phase structure upon contact with an aqueous fluid;

Viewed from a fifth aspect, the invention provides a disposable administration device pre-loaded with a measured dose of a pre-formulation comprising a low viscosity mixture of:

a) at least one neutral lipid (e.g. a diacyl lipid such as a diacyl glycerol);

b) at least one phospholipid;

c) at least one oxygen containing organic solvent; and

d) at least one 5HT₃ antagonist.

Viewed from a sixth aspect the invention provides a kit for the administration of at least one 5HT3 antagonist, said kit containing a measured dose of a formulation comprising a low viscosity mixture of:

a) at least one neutral lipid (e.g. a diacyl lipid such as a diacyl glycerol);

b) at least one phospholipid;

c) at least one oxygen containing organic solvent; and

d) at least one 5HT₃ antagonist.

### DETAILED DESCRIPTION OF THE INVENTION

Highly effective lipid-based controlled-release formulations have been disclosed over the last few years, including formulations such as those of WO2005/117830 which comprise diacyl lipids and phospholipids in appropriate mixtures so as to generate formulations which change phase upon administration. This allows a low-viscosity formulation to be injected and to generate a higher viscosity depot composition in vivo which “traps” the active agent and provides a slow-release effect. Such compositions rely primarily on the lipid matrix to control the active agent release.

It has now been established by the present inventors that 5HT3 antagonists, particularly granisetron and related compounds (salts and structural analogues thereof for example) can be released in a controlled fashion from lipid containing formulations. Such pre-formulations offer advantages to known anti-emetic formulations.

The lipid-based systems described herein comprise lipid components (a) and (b), an organic mono-alcoholic solvent (c), at least one 5HT3 antagonist (d), and optionally a polar solvent (e), and optionally an opioid agonist and/or antagonist (f). Further additives, such as antioxidants, may also be present. In one embodiment a thiolated antioxidant such as mono-thioglycerol may be present. Such additives, such as thiolated antioxidants (e.g. monothioglycerol) may be present at conventional levels, such as less than 5% by weight, e.g. 0.01 to 5% by weight, preferably 0.1 to 2.5% by weight.

Unless where otherwise specified, all amounts and percentages by weight (wt. %) are relative to the pre-formulation. It is the pre-formulation which is administered to the patient. The inventors have previously reported that upon contact with an aqueous body fluid a phase change occurs which converts the low viscosity pre-formulation into a high viscosity liquid crystalline composition, or “depot composition”.

The pre-formulation according to the invention preferably has an L₂ phase structure. Preferably the pre-formulation forms a non-lamellar (e.g. liquid crystalline) phase following administration.

It has been shown previously by the present inventors that when actives such as lidocaine are formulated in a lipid matrix for topical administration, the loading of active agent has a strong effect on the phase formed on exposure to excess aqueous fluid (Barauskas et al. Mol. Pharmaceutics, 2014, 11(3), pp 895-903). The relative proportions of diacyl lipid and phospholipid are also important factors in determining the phase formed. In the case of compositions for topical application, it is important that the liquid crystalline phase formed on administration is bioadhesive. Pre-formulations which form a reversed micellar cubic (Fd3m space group) liquid crystalline phase on exposure to aqueous fluids have been found to be optimal for topical administration.

The pre-formulations of the present invention are preferably suitable for injection and will advantageously undergo a phase change from a low viscosity liquid phase to a higher viscosity phase, such as liquid crystalline phase or L3 “sponge” phase upon exposure to aqueous body fluids. Thus, high loadings of active agents or ratios of lipid components that might disrupt the phase behaviour cannot be expected to function in the formulations of the present invention. The present inventors have, however, unexpectedly established that suitable components and ratios exist to allow for high loadings of 5HT3 antagonists (e.g. 1.6% or greater) as discussed below. It is preferred that on exposure to excess aqueous fluid, the depot formulation formed comprises no less than 50% by weight liquid crystalline phase and/or L3 “sponge” phase. Preferred liquid crystalline phases are reversed micellar cubic (I2) Fd3m phase and reversed hexagonal phase (H2) or combinations thereof. Pre-formulations forming both I2 and H2 liquid crystalline phases, transiently or at equilibrium, are highly preferred. The presence of these and other non-lamellar phases can be determined for instance by small-angle X-ray diffraction (SAXD).

It is preferred that on exposure to excess aqueous fluid, the depot formulation formed comprises no more than 50% by weight, preferably no more than 40%, especially no more than 30% reversed micellar (L2) phase.

It is a feature of the invention that the pre-formulation should be of low viscosity. By this it is meant that the pre-formulation should be injectable through a 19 gauge, preferably a 23 gauge needle under manual pressure. The pre-formulation preferably has a viscosity of 0.1 to 1000 mPas at 20° C., preferably in the range of 100 to 700 mPas at 20° C., such as in the range of 100 to 400 mPas at 20° C.

The pre-formulations of the present invention are generally formulated to be administered parenterally. This administration will generally not be an intra-vascular method but will preferably be subcutaneous (s.c.), intracavitary or intramuscular (i.m.). Typically the administration will be by injection, which term is used herein to indicate any method in which the formulation is passed through the skin, such as by needle, catheter or needle-less (needle-free) injector.

Preferred parenteral administration is by i.m or s.c. injection, most preferably by deep s.c. injection. An important feature of the composition of the invention is that it can be administered both by i.m. and s.c. and other routes without toxicity or significant local effects. It is also suitable for intracavital administration. The deep s.c. injection has the advantage of being less deep and less painful to the subject than the (deep) i.m. injection used for some current depot formulations and is technically most suitable in the present case as it combines ease of injection with low risk of local side effects.

As noted previously, it is a feature of the pre-formulations that they may be administered parenterally, i.e. by injection and must therefore be injectable. By “injectable”, it is meant not only that the pre-formulation is capable of being administered by injection, i.e. of suitably low viscosity, but that the pre-formulation itself is suitable to be administered by injection. The term “injectable” will be understood by the skilled formulator. It implies characteristics of the pre-formulation, including but not limited to: appropriate (i.e. sufficiently low) viscosity, appropriate release profile (e.g., low peak-to-through plasma level ratios), formulation sterility, and appropriate biocompatibility. Although topical formulations comprising a 5HT3 antagonist have previously been described (Barauskas et al. Mol. Pharmaceutics, 2014, 11(3), pp 895-903), these would not be anticipated to meet the requirements of injectable pre-formulations as required of the present pre-formulations, especially in terms of appropriate release profile.

Component a)—Neutral Lipid

Component “a” as indicated herein is a neutral lipid component. The neutral lipid component comprises a polar “head” group and also one or more, preferably two, non-polar “tail” groups. Generally the head and tail portion(s) of the lipid will be joined by an ester moiety but this attachment may be by means of an ether, an amide, a carbon-carbon bond or other attachment. Preferred polar head groups are non-ionic and include polyols such as glycerol, diglycerol and sugar moieties (such as inositol and glucosyl based moieties), sugar derivatives such as sorbitan; and esters of polyols, such as acetate or succinate esters. Preferred polar groups are glycerol and diglycerol, especially glycerol.

In an embodiment component a) of the present invention may comprise at least one ester of a sugar or sugar derivative. Such esters comprise a polar “head” group which is a sugar or sugar derivative, and at least one non-polar “tail” group, preferably a long chain tail group, such as a fatty acid tail group. In this embodiment component a) of the invention may comprise mono-esters, di-esters, tri-esters, tetra-esters or mixtures thereof. Component i) may comprise a mono-ester of a sugar with at least some di-ester of the corresponding sugar.

Examples of polar “head” groups suitable for this embodiment include sugars and sugar derivatives. Examples of sugars include monosaccharides and disaccharides. Examples of derivatives include sugar alcohols such as hexitols and dehydrated sugar alcohols such as hexitans. Dehydrated sugar alcohols are a preferred set of head groups, especially hexitans, especially those derived from allitol, altritol, sorbitol, gulitol, iditol, galactitol and talitol, and cyclised forms thereof. In this embodiment, sorbitan is a particularly suitable head group.

In one aspect, component i) may comprise at least one fatty acid ester of sorbitan. The sorbitan ester comprises a sorbitan head group and at least one non-polar tail group, preferably a lipid-based tail group. Such esters may be mono-, di- or tri-esters and component i) may comprise a mixture of two or more such esters. In one embodiment, component i) will comprise a mixture of mono-, di- and tri-fatty acid esters of a sugar or sugar derivative, especially sorbitan. In all of these sugar esters, the “fatty acid” or “fatty acyl” groups will preferably be the preferred groups referred to herein, such as palmitic, stearic, iso-stearic, oleic and/or linoleic acids. In this embodiment, component i) may comprise, consist or consist essentially of Span™ (available from Croda), which is a mixture of mono-, di- and triacyl sorbitan.

It is especially preferred that component i) is a neutral diacyl lipid, especially a neutral diacyl glycerol. Diacyl glycerols, when present in component a) will comprise glycerol and two acyl chains as indicated herein. Preferred embodiments regarding the structure and nature of these groups and components as indicated herein will apply correspondingly.

In one preferred embodiment, component a) is a diacyl lipid in that it has two non-polar “tail” groups. This is generally preferable to the use of mono-acyl (“lyso”) lipids because these are typically less well tolerated in vivo. The two non-polar groups may have the same or a differing number of carbon atoms and may each independently be saturated or unsaturated. Examples of non-polar groups include C6-C32 alkyl and alkenyl groups, which are typically present as the esters of long chain carboxylic acids. These are often described by reference to the number of carbon atoms and the number of unsaturations in the carbon chain. Thus, CX:Z indicates a hydrocarbon chain having X carbon atoms and Z unsaturations. Examples particularly include caproyl (C6:0), capryloyl (C8:0), capryl (C10:0), lauroyl (C12:0), myristoyl (C14:0), palmitoyl (C16:0), phytanoyl (C16:0), palmitoleoyl (C16:1), stearoyl (C18:0), iso-stearoyl (C18:0), oleoyl (C18:1), elaidoyl (C18:1), linoleoyl (C18:2), linolenoyl (C18:3), arachidonoyl (C20:4), behenoyl (C22:0) and lignoceroyl (C24:9) groups. Thus, typical non-polar chains are based on the fatty acids of natural ester lipids, including caproic, caprylic, capric, lauric, myristic, palmitic, phytanic, palmitolic, stearic, oleic, elaidic, linoleic, linolenic, arachidonic, behenic or lignoceric acids, or the corresponding alcohols. Preferable non-polar chains are palmitic, stearic, oleic and linoleic acids, particularly oleic acid. In one preferred embodiment, component a) comprises components with C16 to C18 alkyl groups, particularly such groups having zero, one or two unsaturations. In particular, component a) may comprise at least 50% of components having such alkyl groups.

In one highly preferred embodiment the diacyl lipid component will comprise, consist essentially of or consist of at least one diacyl glycerol (DAG), thus having two non-polar “tail” groups. The two non-polar groups may have the same or different and may be any of the groups indicated above.

Mixtures of any number of diacyl lipids may be used as component a). Preferably this component will include at least a portion of C18 lipids (e.g. DAG having one or more C18:0, C18:1, C18:2 or C18:3 non-polar groups), such as glycerol dioleate (GDO) and/or glycerol dilinoleate (GDL). A highly preferred example is DAG comprising at least 50%, preferably at least 80% and even comprising substantially 100% GDO.

Since GDO and other diacyl glycerols are products derived from natural sources, there is generally a certain proportion of “contaminant” lipid having other chain lengths etc. In one aspect, GDO as used herein is thus used to indicate any commercial grade of GDO with concomitant impurities (i.e. GDO of commercial purity). These impurities may be separated and removed by purification but providing the grade is consistent and the properties predictable, this is rarely necessary. If necessary, however, “GDO” may be essentially chemically pure GDO, such as at least 80% pure, preferably at least 85% pure and more preferably at least 90% pure GDO. Corresponding purities apply to other components indicated herein.

An alternative or additional highly preferred class of compounds for use as all or part of component a) are tocopherols. As used herein, the term “a tocopherol” is used to indicate the non-ionic lipid tocopherol, often known as vitamin E, and/or any suitable salts and/or analogues thereof. Suitable analogues will be those providing the phase-behaviour, lack of toxicity, and phase change upon exposure to aqueous fluids, which characterise the compositions of the present invention. Such analogues will generally not form liquid crystalline phase structures as a pure compound in water. The most preferred of the tocopherols is tocopherol itself, having the structure below. Evidently, particularly where this is purified from a natural source, there may be a small proportion of non-tocopherol “contaminant” but this will not be sufficient to alter the advantageous phase-behaviour or lack of toxicity. Typically, a tocopherol will contain no more than 10% of non-tocopherol-analogue compounds, preferably no more than 5% and most preferably no more than 2% by weight.

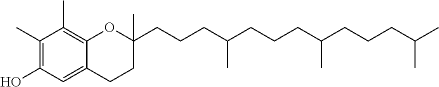 [SMILES: Cc1c(O)cc2c(c1C)OC(C)(CCCC(C)CCCC(C)CCCC(C)C)CC2]

### Tocopherol (Vitamin E)

In all embodiments, preferable levels for component a) are 20-54 wt. %, preferably 20-45 wt. %, more preferably 25-40 wt. %. This range should be interpreted as the combined weight of all neutral lipids (e.g. diacyl lipids such as diacylglycerol(s)) present in the pre-formulation relative to the weight of the entire pre-formulation. These ranges are particularly appropriate where component a) comprises, consists or consists essentially of diacyl glycerol(s).

Ratios by weight of a):b) are typically 40:60 to 64:36, such as 40:60 to 62:38 or 40:60 to 60:40, preferably 45:55 to 55:45 and more preferably 40:60 to 54:46. Ratios of around 50:50 (i.e. 48:52 to 52:48) are highly effective.

Component b)—Phospholipid

Component “b” in the lipid matrices of the present invention is at least one phospholipid. As with component a), this component comprises a polar “head” group and at least one non-polar “tail” group. The difference between components a) and b) lies principally in the polar group. The non-polar portions may thus suitably be derived from the fatty acids or corresponding alcohols considered above for component a). As with component a), the phospholipid will preferably comprise two non-polar groups. In particular C16 to C18 acyl groups having zero, one or two unsaturations are highly suitable as moieties forming the non-polar group of the compounds of component b). It will typically be the case that the phospholipid will contain two non-polar groups, although one or more constituents of this component may have one non-polar moiety. Where more than one non-polar group is present these may be the same or different.

Preferred phospholipid polar “head” groups include phosphatidylcholine (PC), phosphatidylethanolamine (PE), sphingomyelin (SM), phosphatidylserine (PS), and/or phosphatidylinositol (PI). Most preferred is PC. It is particularly preferred that component b) comprises at least 50 wt. % PC, more preferably greater than 50% PC, such as at least 52% (e.g. 52 to 99%), at least 55% or at least 60% by wt. PC, more preferably at least 70 wt. % PC, especially more than 80 wt. % PC, particularly more than 90 wt. % PC.

In one embodiment applicable to all aspects of the invention, component b) comprises PC. Preferably the PC is derived from soy, although other sources, including purified and/or synthetic dioleoyl PC (DOPC) may be used. Preferably the PC comprises 18:2 fatty acids as the primary fatty acid component (e.g. at least 51%) with 16:0 and/or 18:1 as the secondary fatty acid components (e.g. greater than 3% but less than 40%). These are preferably present in the PC at a ratio of between 1.5:1 and 6:1. PC having approximately 60-65% 18:2, 10 to 20% 16:0, 5-15% 18:1, with the balance predominantly other 16 carbon and 18 carbon fatty acids is preferred and is typical of soy PC.

The phosphatidyl choline portion, even more suitably than any diacyl glycerol portion, may be derived from a natural source. Suitable sources of phospholipids include egg, heart (e.g. bovine), brain, liver (e.g. bovine) and plant sources including soybean. Such sources may provide one or more constituents of component b, which may comprise any mixture of phospholipids. Any single PC or mixture of PCs from these or other sources may be used, but mixtures comprising soy PC or egg PC are highly suitable. The PC component preferably contains at least 50% soy PC or egg PC, more preferably at least 75% soy PC or egg PC and most preferably essentially pure soy PC or egg PC.

In an alternative but equally preferred embodiment, the PC component may comprise purified synthetic dioleoyl PC (DOPC) and/or palmitoyl oleoyl PC (POPC). The use of synthetic PC may provide increased stability and so will be particularly preferable for compositions needing to be stable to long term storage, and/or having a long release period in vivo. In this embodiment the PC component preferably contains at least 50% synthetic DOPC and/or POPC, more preferably at least 75% synthetic DOPC and/or POPC and most preferably essentially pure synthetic DOPC or POPC. Any remaining PC is preferably soy or egg PC as above.

Since the pre-formulations of the invention are to be administered to a subject for the controlled release of at least one active agent, it is important that the components are biocompatible. In this regard, the preferred lipid matrices for use in the pre-formulations of the present invention are highly advantageous since both phospholipids (e.g. PC) and neutral diacyl lipids (e.g. DAGs) are well tolerated and are broken down in vivo into components that are naturally present in the mammalian body.

Synthetic or highly purified PCs, such as dioleoyl phosphatidy choline (DOPC) are highly appropriate as all or part of component b). The synthetic dioleoyl PC is most preferably 1,2-dioleoyl-sn-glycero-3-phosphocholine, and other synthetic PC components include DDPC (1,2-Didecanoyl-sn-glycero-3-phosphocholine); DEPC(1,2-Dierucoyl-sn-glycero-3-phosphocholine); DLOPC(1,2-Dilinoleoyl-sn-glycero-3-phosphocholine); DLPC(1,2-Dilauroyl-sn-glycero-3-phosphocholine); DMPC(1,2-Dimyristoyl-sn-glycero-3-phosphocholine); DOPC(1,2-Dioleoyl-sn-glycero-3-phosphocholine); DPPC(1,2-Dipalmitoyl-sn-glycero-3-phosphocholine); DSPC(1,2-Distearoyl-sn-glycero-3-phosphocholine); MPPC(1-Myristoyl-2-palmitoyl-sn-glycero 3-phosphocholine); MSPC(1-Myristoyl-2-stearoyl-sn-glycero-3-phosphocholine); PMPC(1-Palmitoyl-2-myristoyl-sn-glycero-3-phosphocholine); POPC(1-Palmitoyl-2-oleoyl-sn-glycero-3-phosphocholine); PSPC(1-Palmitoyl-2-stearoyl-sn-glycero-3-phosphocholine); SMPC(1-Stearoyl-2-myristoyl-sn-glycero-3-phosphocholine); SOPC(1-Stearoyl-2-oleoyl-sn-glycero-3-phosphocholine); and SPPC(1-Stearoyl-2-palmitoyl-sn-glycero-3-phosphocholine), or any combination thereof.

In some circumstances, such as the absence of preserving agents such as EDTA, the use of synthetic or highly purified PCs (e.g. DOPC) may provide greater stability for the active agent in the formulations. Thus in one embodiment, component b) may comprise (e.g. may comprise at least 75%) synthetic or highly purified (e.g. purity >90%) PCs (e.g. DOPC). This may particularly be in the absence of chelating agents such as EDTA. In an alternative embodiment, component b) may comprise (e.g. comprise at least 75%) naturally derived PCs, such as soy PC or egg PC. This will particularly be where at least one stabilising component (such as an antioxidant, chelator etc) is included in the precursor formulation.

A particularly favoured combination of components a) and b) are GDO with PC, especially GDO with soy PC and/or DOPC. Appropriate amounts of each component suitable for the combination are those amounts indicated herein for the individual components in any combination. This applies also to any combinations of components indicated herein, where context allows.

Preferable levels for component b) are 20-60 wt. %, preferably 25-50 wt. %, more preferably 25-45 wt. %. This range should be interpreted as the combined weight of all phospholipid(s) present in the pre-formulation relative to the weight of the entire pre-formulation.

Component c)—Oxygen Containing Organic Solvent

Component c) of the pre-formulations of the invention is an organic oxygen-containing solvent, preferably a mono-alcoholic solvent. Since the pre-formulation is to generate a depot composition following administration (e.g. in vivo), typically upon contact with excess aqueous fluid, it is desirable that this solvent be tolerable to the subject and be capable of mixing with the aqueous fluid, and/or diffusing or dissolving out of the pre-formulation into the aqueous fluid. Solvents having at least moderate water solubility are thus preferred.

Most preferably component c) comprises or consists of at least one mono-alcoholic solvent, sulfoxide or amide. Especially preferred mono-alcoholic solvents are ethanol, propanol, iso-propanol, and benzyl alcohol or mixtures thereof. An especially preferred sulfoxide is dimethylsulfoxide. A particularly preferred amide is N-methyl-pyrrolidone (NMP). Most preferably component c) comprises or consists of at least one solvent selected from ethanol, DMSO and/or NMP. Most preferably component c) comprises or consists of ethanol.

In a preferred embodiment, the solvent is such that a relatively small addition to a mixture comprising a) and b) (i.e. preferably below 15%) gives large viscosity reductions, of one order of magnitude or more. As described previously by the present inventors, the addition of 10% organic mono-alcohol solvent can give a reduction of two or more orders of magnitude in viscosity over the solvent-free composition, or over a depot containing only a polar solvent such as water, or glycerol.

The amount of component c) in the pre-formulation will have a considerable effect upon several features. In particular, the viscosity and the rate (and duration) of release will alter significantly with the solvent level. The amount of solvent will thus be at least sufficient to provide a low viscosity mixture but will additionally be determined so as to provide the desired release rate. This may be determined by routine methods, for instance as set out in WO2012/160213. Typically a level of 0.1 to 35 wt. %, particularly 1 to 30 wt. %, particularly 5 to 25 wt. % solvent will provide suitable release and viscosity properties. This will preferably be 5 to 20 wt. % and an amount of around 15 wt. % (e.g. 15±2 wt. %) is highly effective.

As indicated above, the amount of component c) in the pre-formulations of the invention will be at least sufficient to provide a low viscosity mixture (e.g. a molecular solution or other low-viscosity phase as described herein) of components a), b), c) and d), and optionally e) and/or f) and will be easily determined for any particular combination of components by standard methods.

The phase behaviour may be analysed by techniques such as visual observation in combination with polarized light microscopy, X-ray scattering and diffraction techniques, nuclear magnetic resonance, and cryo-transmission electron microscopy (cryo-TEM) to look for solutions, L₂ or L₃ phases, or liquid crystalline phases or as in the case of cryoTEM, dispersed fragments of such phases. Viscosity may be measured directly by standard means. As described above, an appropriate practical viscosity is that which can effectively be syringed and particularly sterile filtered. This will be assessed easily as indicated herein.

A highly preferred combination for components a), b) and c) is soy PC, GDO and ethanol. As indicated above, appropriate amounts of each component suitable for the combination are those amounts indicated herein for the individual components, in any combination.

It is preferable that little or none of component c) contains halogen substituted hydrocarbons since these tend to have lower biocompatibility.

Component c) as used herein may be a single solvent or a mixture of suitable solvents but will generally be of low viscosity. This is important because one of the preferred aspects of the present invention is that it provides pre-formulations that are of low viscosity and one role of a suitable solvent is to reduce this viscosity. This reduction will be a combination of the effect of the lower viscosity of the solvent and the effect of the molecular interactions between solvent and lipid composition. One previous observation of the present inventors, as described in WO2012/160213, is that the oxygen-containing solvents of low viscosity described herein have highly advantageous and unexpected molecular interactions with the lipid parts of the composition, thereby providing a non-linear reduction in viscosity with the addition of a small volume of solvent.

The viscosity of the “low viscosity” solvent component c) (single solvent or mixture) should typically be no more than 18 mPas at 20° C. This is preferably no more than 15 mPas, more preferably no more than 10 mPas and most preferably no more than 7 mPas at 20° C.

Component d)—5HT3 Antagonist

As mentioned previously, 5HT3 antagonists are known to be particularly effective anti-emetics. It is known that the interaction of seretonin with various 5HT3 receptors is responsible for initiating the vomiting reflex. 5HT3 receptor antagonists suppresses this response by binding to the 5HT3 receptor and blocking the action of other binders such as seretonin.

The 5HT3 antagonist present in pre-formulations of the invention may be a first or second generation 5HT3 antagonist. Preferably this is selected from ondansetron, tropisetron, granisetron, dolasetron, palonosetron, alosetron, cilansetron and/or ramosetron or mixtures thereof.

A particularly preferred 5HT3 antagonist particularly preferred in all aspects of the invention is granisetron, having the structure below:

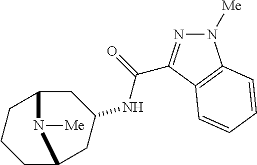

### Granisetron

Pre-formulations of the invention may comprise the 5HT3 antagonist as the free base or a salt thereof. Salt forms of the 5HT₃ antagonist are preferred.

Salts of the 5HT₃ antagonist will comprise a cation of the 5HT₃ antagonist and at least one pharmaceutically acceptable anion, such as a halide, pamoate, citrate or tartrate anion. Particularly preferred salts include the chloride, citrate, pamoate and tartrate salts. Most preferably component d) comprises or consists of granisetron, or a biologically acceptable salt of granisetron. A particularly preferred salt, applicable to all embodiments is granisetron chloride.

Conditions which are suitable for treatment or prophylaxis using pre-formulations of the invention include emesis, nausea, vomiting, chemotherapy and/or radiotherapy and/or endoradionuclide therapy induced nausea and vomiting, post-operative and extended post-operative nausea and vomiting, pain, post-operative and extended post-operative pain, delayed nausea and vomiting in patients undergoing chemotherapy including HEC and MEC, motion sickness, IBS, gastroenteritis and/or related conditions.

As will be explained in more detail herein, depending on the condition for which treatment or prophylaxis is intended, depot compositions and the corresponding precursor formulations of the invention may be formulated such that they release the active agent(s) over a particular time period. This may be achieved by varying the proportions and nature of components a)-d) and optionally components e) and f) (as described below) if present in the pre-formulation.

Where the condition for which treatment or prophlaxis is intended only requires therapeutically effective amounts of a 5HT3 receptor antagonist for a short period, the depot precusor may be formulated so as to generate an in-vivo depot which will release the active agent(s) over a period of a few days, such as less than 72 hours, less than 48 hours, such as about 24 hours. In such cases the depot composition may release the active agent(s) over a period of 24-72 hours. A level which is “therapeutically effective” is used herein to indicate that the plasma concentration of the API is above the minimum therapeutic concentration for that API. Accordingly, a depot which provides a therapeutically effective amount of active agent for 24-72 hours will provide a plasma concentration above the minimum therapeutic level of the API in the subject for a period of 24-72 hours on average.

Conditions in which the depot composition may be required to release its active agent(s) over a period of 72 hours or less include treatment or prophylaxis against: non-induced nausea and non-induced vomiting (i.e. where these conditions are not side-effects of other medical treatment), nausea and vomiting in patients undergoing HEC, MEC, radiotherapy or endoradionuclide therapy, post-operative nausea and vomiting, post-operative pain, motion sickness, IBS, gastroenteritis and/or related conditions.

Alternatively, the depot composition might be required to provide a sustained release of active agent(s) for a longer period. For instance, for some conditions it may be advantageous to provide a depot composition which can release a therapeutically effective amount of active agent(s) over a period of up to 28 days, such 1 to 21 days, in some instances 1-14 days, or 1-7 days. A depot composition releasing its active agent(s) over a period of 3-21 days (i.e. providing a therapeutically effective amount of API for 3-21 days), 3-14 days or 3-7 days is envisaged for certain applications.

Conditions in which the depot compositions may be required to provide a sustained release of active agent(s) for a longer period, i.e. 3-21 days as described above, include in the treatment or prophylaxis against: extended post-operative pain, extended post-operative nausea and vomiting, acute and delayed chemotherapy-induced nausea and vomiting (CINV) in patients undergoing chemotherapy including HEC and MEC, radiotherapy or endoradionuclide therapy, and/or related conditions.

Doses of the 5HT₃ antagonist suitable for inclusion in the formulation, and thus the volume of formulation used, will depend upon the release rate and release duration, as well as the desired therapeutic level, the activity of the specific 5HT3 antagonist, and the rate of clearance of the particular active chosen. A suitable 5HT3 antagonist for use in all aspects of the invention is granisetron.

Unless where otherwise specified, the percentage by weight of 5HT3 antagonist refers to that calculated in terms of the free base. The 5HT3 antagonist will typically be present in an amount of 0.1 to 25 wt. % of the pre-formulation, preferably 0.5 to 15 wt. %, especially 1 to 15 wt. %, such as 1 to 10 wt. %. These ranges are particularly preferred loadings for granisetron. In all embodiments it is preferred that the loading of 5HT3 antagonist in the pre-formulation is greater than 1.5 wt. %, preferably ≥1.6 wt. %. A level of 2.0 to 5.0 wt % is particularly preferred in some embodiments. The loading of 5HT3 antagonist will of course depend on the duration of the depot product and the condition to be treated. All loadings for 5HT3 antagonists are calculated herein as the free base unless indicated otherwise.

Typically an amount of 1 to 200 mg 5HT3 antagonist per dose would be suitable for providing a therapeutic level for between 1 and 28 days (calculated as the amount of 5HT3 antagonist free base). This will preferably be 1 to 100 mg, especially between 1 to 50 mg. For granisetron, the level will typically be around 1 to 100 mg, such as 1 to 50 mg or 2-40 mg, especially 3 to 30 mg or 5-20 mg (e.g. for a 1 to 28 day or 1 to 7 day duration).

Preferably, the amount of granisetron will be around 0.2 to 3 mg per day between injections, preferably 2±0.5 mg per day (or 2.1±0.5 mg per day), for depots designed for release over 1 to 28 days, preferably for release over 1 to 14 days such as 1 to 7 days. Evidently, the stability of the active and linearity of the release rate will mean that the loading to duration may not be a linear relationship. A depot administered every 5 days might have, for example 5 to 12.5 mg (e.g. 7 to 12.5 mg) or a 7 day depot have 10.5 to 18 mg of active (e.g. 12 to 18 mg), especially granisetron.

Depot formulations for use in treating CINV may be formulated to contain 4 to 20 mg of granisetron for a 3-7 day duration. The corresponding pre-formulations may comprise 4 to 20 mg of granisetron, such as 6 to 18 mg, 8 to 16 mg, or 10 to 14 mg.

The absolute levels of the 5HT3 antagonist specified above are particularly suitable for a human subject.

In general, for a pre-formulation forming a depot having a release duration of 1 to 7 days, an appropriate dosage of 5HT3 antagonist for a given mammalian (preferably human) subject is 0.05 to 30 mg/kg, preferably 0.05 to 15 mg/kg, such as 0.1 to 10 mg/kg. These levels are particularly appropriate for granisetron.

Component e)—Polar Solvent

The present inventors have established that for certain compositions of the invention, the use of an alcohol solvent in combination with a polar solvent such as a diol or water allows a significant improvement in the performance of certain lipid-based controlled-release compositions. In particular, the addition of a diol, such as propylene glycol, or water has been observed to reduce the viscosity of a lipid/alcohol/active agent formulation without adversely affecting the release profile of the active agent and/or allows the proportion of alcohol to be increased without adversely affecting the release profile and/or allows an improvement in the release profile. The polar solvent may also allow for higher loading levels of the active agent (e.g. component d) and/or optionally f) without disrupting the phase behaviour of the depot upon exposure to aqueous fluid. By “adversely affecting the release profile” is intended to indicate that the ratio of Cmax/Cave is increased and/or the ratio of Cmax/Cmin is increased (for example increased by a factor of at least 1.2). Similarly an improvement in the release profile indicates that the ratio of Cmax/Cave and/or Cmax/Cmin is decreased (e.g. decreased by a factor of at least 1.2.). The presence of a polar solvent may also allow for suitable drug loading levels, particularly while maintaining desirable phase behaviour, as described herein.

Although it has previously been suggested that lipid controlled-release compositions should be formulated substantially in the absence of water, in order to avoid the conversion to high-viscosity liquid crystalline phases, it has also been established that a small and carefully controlled amount of a polar solvent such as water can provide considerable benefits. In particular, the inclusion of a polar solvent (preferably comprising water) has been shown to allow further benefits including tuning of the initial release of active agent, improvements to the loadings of some active agents including various salts of active agents, and may provide faster depot formation. Any one of these factors potentially provides a significant improvement in the context of therapeutic drug delivery, patient health and/or patient compliance.

In the context of the present invention, the inventors have established that the use of a salt of a 5HT₃ antagonist in combination with a lipid delivery formulation as described herein provides particular advantages when that formulation comprises a polar solvent as described herein. In particular, the combination of a salt of a 5HT₃ antagonist and a pre-formulation of the invention comprising component e) as described herein may provide a pre-formulation that generates a depot having a significantly extended duration of release. Since the pre-formulations remain of low-viscosity and come into contact with water upon injection in any case, the significant advantage of combining a salt of a 5HT₃ antagonist with a pre-formulation including component e) cannot readily be anticipated. The advantages of this combination are illustrated in the Examples herein below and the attached Figures. Without being bound by theory, it is believed that the addition of a polar solvent causes a change in the microscopic structure of the pre-formulation, possibly to provide domains of polar and non-polar nature. This may help stabilise the highly polar 5HT₃ antagonist salt and thereby retard release.

Preferred 5HT₃ antagonist salts include all of those discussed herein, such as halide salts, particularly bromide or chloride, and most preferably chloride. Granisetron chloride is a highly preferred example.

Anti-emetics may be administered for the treatment or for prophylaxis of a wide range of conditions including emesis, nausea, vomiting, chemotherapy and/or radiotherapy and/or endoradionuclide therapy induced nausea and vomiting, post-operative and extended post-operative nausea and vomiting, pain, post-operative and extended post-operative pain, acute and delayed nausea and vomiting in patients undergoing chemotherapy including HEC and MEC, cancer, motion sickness, IBS, gastroenteritis and/or related conditions.

The duration of the depot composition will depend on which condition is being treated or prophlaxed against. This can be thought of as “short duration” in the case of depot compositions releasing their active agent(s) over a period of 24-72 hours, and “extended duration” where the release is over a period of 3-28 days.

In conditions which require a “short duration” depot product, the Cmax produced by the depot product will generally be higher than the Cmax of an extended duration product. In the case of “extended” depot products, the patient will typically be exposed to a lower peak concentration (Cmax) of the active substance, but for a longer duration.

The present inventors have now established that the inclusion of a polar solvent in pre-formulations comprising a 5HT3 antagonist (particularly a salt thereof as discussed herein) results in the production of longer-release depots relative to when the polar solvent is absent. The duration of the release of the 5HT3 antagonist can therefore be tuned to be compatible with the nature of treatment in question by varying the amount of polar solvent included in the pre-formulation.

Pre-formulations of the present invention may thus also contain a polar solvent, component e). When present, a suitable amount will typically be greater than 1% by weight of the pre-formulation, for example 5-35 wt. %, particularly 8-30 wt. %, especially 10-30 wt. %. Component c) may be present in the range 8-20 wt. %, especially 15 wt. %±5 wt. %.

Component e) is preferably water, propylene glycol or mixtures thereof. In one preferred aspect, the pre-formulations of the invention contain ethanol as component c) with water and/or propylene glycol as component e). Component e) may comprise or consist of water. Alternatively, component e) may comprise or consist of propylene glycol. A particularly preferred combination are pre-formulations in which component c) comprises, consists essentially of, or consists of ethanol, and component e) comprises, consists essentially of, or consists of water.

Preferably the total level of components c) and e) is not more than 50 wt. %, preferably not more than 40 wt. %, more preferably not more than 35 wt. %, preferably 10-40 wt. %, most preferably 10-35 wt. %. This range should be interpreted as the combined weight of components c) and e) present in the pre-formulation relative to the weight of the entire pre-formulation.

The ratio of components c) and e) will also have potential advantages in the compositions of the invention. In particular, by inclusion of some polar solvent (especially water) which is miscible with the mono-alcohol component, the slight sensation that may be caused at the injection site from the alcohol content can be substantially eliminated. Thus, in one embodiment, the ratio of components c):e) (w/w) may be in the range 30:70 to 70:30, more preferably 40:60 to 60:40. Ratios of c):e) ranging from 50:50 to 70:30 (especially for ethanol:water) are thus appropriate in one embodiment. Approximately equal amounts of components c) and e) are highly appropriate. Where component e) consists of or consists essentially of water, the ratio c)/e) is preferably ≥1.

A highly preferred combination for the lipid matrix aspect is soy PC, GDO, ethanol, and water/propylene glycol or mixtures thereof. An even further preferred combination for the lipid matrix aspect is soy PC, GDO, ethanol and water. These delivery systems may be used advantageously with salts of 5HT3 antagonists, particularly halide salts, such as chloride salts (including granisetron chloride). As indicated above, appropriate amounts of each component suitable for the combination are those amounts indicated herein for the individual components, in any combination.

Component f)—Opioid Agonist and/or Antagonist

The administration of an opioid receptor agonist and/or antagonist (“opioid”), for instance as an anaesthetic or during treatment or maintenance therapy for opioid addiction, may cause nausea and vomiting as side-effects. Opioids are also used heavily in situations where emesis may be expected as a side effect of a disease or treatment, such as in surgery and/or cancer treatment, including all of the treatments and conditions discussed herein, where appropriate. The present inventors have now established that by administering the opioid agonist and/or antagonist in combination with an anti-emetic in a controlled release matrix, it is possible to reduce or eliminate these side-effects of the opioid, disease or treatment. This is preferably in combination with the benefits of the opioid for pain relief or the treatment or maintenance of symptoms of opioid withdrawal. The opioid and anti-emetic are preferably combined in a single pre-formulation and administered together, rather than being administered via separate injections. This allows for a sustained and tapering profile of both opioid and anti-emetic, which is an appropriate profile in many situations. The combined medicament also allows for ease of administration and good patient compliance.

The inventors have established that there are several advantages to administering an opioid in combination with an anti-emetic. Firstly, administration of the anti-emetic at the same time as the opioid agonist and/or antagonist may reduce or substantially eliminate the symptoms of nausea and vomiting. Secondly, since the release rates of both the anti-emetic and the opioid agonist and/or antagonist are controlled by the slow-release matrix, the relative amounts of each component released by the depot product are synchronised, in the sense that both the initial opioid release rate and the anti-emetic release rate are highest shortly after administration, when the need for anti-emesis and analgesia will be highest in many cases. Similarly, after a certain duration the opioid release of the depot will be lower and the release of anti-emetic from the depot will likewise be lower corresponding to a recovery of the subject from the underlying cause, such as surgery or a dose of treatment. A depot product containing both an opioid agonist and/or antagonist in addition to an anti-emetic therefore avoids the patient experiencing levels of anti-emetic which are beyond what is needed to prevent the sensation of nausea and simultaneously gives a tapering profile of analgesia.

It will be appreciated that the measured initial release profiles (in plasma) of the anti-emetic and the opioid agonist and/or antagonist might not be exactly synchronised since these components have different properties and therefore the time to reach Cₘₐₓ may be different for both. However, as shown in the examples below, typically within 24 hours the anti-emetic and the opioid have both achieved Cₘₐₓ and show a synchronized, tapering release profile. In one preferred embodiment the maximum plasma level (Cmax) of the 5HT3 antagonist is reached within 24 hours after injection, preferably within 12 hours. If present, the maximum plasma level (Cmax) of the opioid agonist and/or antagonist is also preferably reached within 24 hours after injection, preferably within 12 hours.

A particularly preferred opioid having both agonistic and antagonistic properties is buprenorphine. Buprenorphine free base, or any biologically acceptable salt of buprenorphine may be used in all aspects of the invention. However, buprenorphine free base is most preferred. A particularly preferred 5HT3 antagonist is granisetron. Especially preferred are pre-formulations comprising or consisting of granisetron as component d), and comprising or consisting of buprenorphine as component f). Most preferred is a combination of buprenorphine free base and granisetron salt, such as halide salt, especially chloride.

In an embodiment the ratio of 5HT3 antagonist:opioid ((d):(f)) may range from 5:95 to 95:5 (w/w), such as from 15:85 to 85:15 or 25:75 to 75:25. In one aspect the relative amounts of 5HT3 antagonist (% by weight) is greater than the amount of opioid, i.e. the ratio of (d):(f) is from 51:49 to 95:5, such as 55:45 to 95:5 or 60:40 to 90:10.

The opioid agonist(s) and/or antagonist(s) comprising component (f) may be present in an amount of up to 10 wt. % of the pre-formulation. When present, suitable levels may be 0.1-10 wt. % of the pre-formulation, such as 0.1-8 wt. % of the pre-formulation, especially 0.2 to 4.0 wt %. These levels are also applicable to the sum total amounts of opioid agonists or antagonists where more than one opioid agonist or antagonist is present as component (f). As with the 5HT3 antagonist, amounts of opioid agonist and/or antagonist are calculated herein as the free base unless indicated otherwise. Ratios will evidently be calculated likewise.

The loading of opioid in the pre-formulation and depot product will depend on the condition being treated. However, for the treatment or prophylaxis against post-operative and extended post-operative pain, or the treatment or prophylaxis against post-operative or extended post-operative nausea and vomiting, or the treatment against acute and delayed nausea and vomiting in patients undergoing chemotherapy including HEC and MEC, a level of 0.5 to 30 mg opioid (calculated as opioid free base) is appropriate, especially preferred are loadings of 0.5 to 20 mg or 0.5 to 12 mg per dose, i.e. per injection. These levels are particularly preferred for buprenorphine.

For a product having a release duration of 1 to 7 days, an appropriate level of opioid is 0.2 to 3 mg opioid per day, preferably 0.5 to 2 mg per day. Accordingly, a product having a release duration of 1-3 days may comprise 0.2 to 9 mg of opioid, preferably 0.5 to 6 mg of opioid. A product having a release duration of 3-7 days may comprise 0.6 to 21 mg, preferably 1.5 to 14 mg of opioid. A product having a release duration of 5-7 days may comprise 1 to 21 mg, preferably 2.5 to 14 mg of opioid. These levels are particularly appropriate for buprenorphine. It will be appreciated that these levels are applicable to the pre-formulation, which forms the depot product on contact with excess aqueous fluid.

The absolute levels of opioid specified above are particularly suitable for a human subject.

In general, for a depot having a release duration of 1 to 7 days, an appropriate dosage of opioid for a given mammalian (preferably human) subject is 0.0003 to 1.5 mg/kg (e.g. 0.003 to 0.15 or 0.03 to 1.5 mg/kg), preferably 0.01 to 1 mg/kg, such as 0.1 to 0.8 mg/kg. These levels are particularly appropriate for buprenorphine.

The term “post-operative treatment” refers to a depot composition which provides for the release of a therapeutically effective amount of active agent(s) for 24-72 hours following administration. “Extended post-operative” refers to a depot composition which provides for the release of a therapeutically effective amount of active agent(s) for 3-28 days following administration, such as 3-14 days or 3-7 days.

As detailed in relation to the polar solvent component e), the duration of the depot product may be varied by adjusting the level of polar solvent(s) in the pre-formulation. In particular, as indicated in the Figures, low levels of polar solvent may produce a depot product having a shorter duration and suitable for the treatment or prophylaxis against post-operative pain or post-operative nausea and vomiting, whereas pre-formulations comprising a larger proportion of polar solvent may produce a depot product having a longer duration, suitable for the treatment or prophylaxis against extended post-operative pain, or extended post-operative nausea and vomiting.

A single pre-formulation with a suitably chosen level of opioid, 5HT3 antagonist and optionally polar solvent may be used to treat both short-term and extended post-operative pain. Alternatively, two separate pre-formulations may be administered; one to treat short-term post-operative pain and another to treat extended post-operative pain.

It is envisaged that depot products providing for the release of a therapeutic amount of a 5HT3 antagonist and an opioid agonist and/or antagonist over a 24-72 hour period, will generally result in a higher peak plasma concentration (Cmax) than a depot which provides for the release of active agent(s) over a 3-28 day period. In some embodiments therefore it may be desirable to minimise the effects experienced by the patient associated with high opioid levels. It may also be desirable to protect against the possibility of overdose using such depots, i.e. through drug diversion.

This can be achieved by the inclusion of naloxone in addition to the 5HT3 antagonist and other opioid agonist and/or antagonist. It is known that naloxone can be combined with other opioids to block the effect of the opioid at high doses.

In one embodiment, the pre-formulation may comprise a 5HT3 antagonist as component d), and both naloxone and another opioid agonist and/or antagonist as component f). A particularly preferred combination for certain embodiments of the invention is granisetron as component d), and buprenorphine and naloxone as component f). Such pre-formulations are particularly suitable for the treatment or prophylaxis against post-operative pain, and post-operative nausea and vomiting.

When both are present, the ratio of naloxone (free base):opioid agonist and/or antagonist (free base) is 1:7 to 1:2 (w/w), preferably 1:6 to 1:3. These ratios are particularly suitable where component f) consists of buprenorphine and naloxone.

Pre-formulations comprising a 5HT3 antagonist as component d), and both naloxone and an opioid and/or antagonist as component f) preferably provide a therapeutically effective release of active agents for 24-72 hours. Preferred proportions of d) and f) in this embodiment are as described in the respective sections above.

In another embodiment, slow-release formulations of the invention may be used in opioid management or opioid withdrawal. This typically involves providing a patient with regular administration (e.g. weekly) of opioid, at a gradually tapering level. Nausea and vomiting are also common side-effects of opioid management.

In this embodiment the dose of buprenorphine present in the pre-formulation (and the corresponding slow-release depot) will typically be higher than for depots suitable for treating post-operative and extended post-operative pain. The depot will also be formulated to provide an effective release of buprenorphine and 5HT3 antagonist for a period of 5-14 days, especially around 7 days.

In this embodiment suitable levels of buprenorphine will be 1 to 5 mg/day between injections, preferably 1 to 4.6 mg/day between injections. A pre-formulation for use in opioid management will therefore comprise an amount of 7 to 35 mg of buprenorphine, preferably 7 to 32 mg, for a depot having a release duration of 7 days.

In this embodiment suitable levels of 5HT3 antagonist (especially granisetron) will be 0.5 to 3 mg/day between injections. A pre-formulation for use in opioid management will therefore comprise an amount of 3.5 to 21 mg, preferably 5 to 15 mg of 5HT3 antagonist for a depot having a release duration of 7 days.

The Invention will now be further illustrated by reference to the following non-limiting Examples and the attached Figures.

### FIGURES

FIG. 1 demonstrates the mean plasma concentration of granisetron for Formulation 1A and Formulation 1B. Error bars denote standard deviation (n=6).

FIG. 2 demonstrates the mean plasma concentration of granisetron for Formulation 2A-2D. Error bars denote standard deviation (n=6).

FIG. 3 shows SAXD results (25, 37 and 42° C.) from samples of fully hydrated Lipid/EtOH/WFI mixtures prepared at SPC/GDO weight ratio 50/50 and EtOH/WFI weight ratio 15/10 as a function of GRN(Cl) concentration (between 0 and 4 wt %) and temperature.

FIG. 4 shows SAXD results (25, 37 and 42° C.) from samples of fully hydrated Lipid/EtOH/WFI mixtures prepared at SPC/GDO weight ratio 35/65 and EtOH/WFI weight ratio 15/10 as a function of GRN(Cl) concentration (between 0 and 4 wt %) and temperature.

FIG. 5 shows the mean plasma concentration of granisetron in rat for Formulation 7A-7B. Error bars denote standard deviation (n=6).

FIG. 6 shows the mean plasma concentration of buprenorphine in rat for Formulation 7B. Error bars denote standard deviation (n=6).

FIG. 7 shows the mean plasma concentration of granisetron in dog for Formulation 7A-7B. Error bars denote standard deviation (n=6).

FIG. 8 shows the mean plasma concentration of buprenorphine in dog for Formulation 7B. Error bars denote standard deviation (n=6).

### EXAMPLES

### Example 1

Pre-formulations (5 g) of granisetron (GRN) and granisetron hydrochloride (GRN(Cl)) were produced by weighing all components (Table 1) in one vial followed by mixing by end-over-end rotation at ambient temperature until clear and homogenous solutions were obtained. The resulting formulations were filtered through sterile 0.22 μm syringe filters under nitrogen pressure.

TABLE 1
Formulation compositions (wt %).

| Formulation | GRN | GRN(Cl) | GDO | SPC | BzOH | EtOH | WFI |
|---|---|---|---|---|---|---|---|
| 1A | 4.0 | — | 34.2 | 34.2 | 20.0 | 7.6 | — |
| 1B | — | 4.0 | 32.6 | 32.6 | — | 15.8 | 15.0 |

Abbreviations:
GRN = granisetron;
GRN(Cl) = granisetron hydrochloride;
GDO = glycerol dioleate;
SPC = soy phosphatidylcholine;
BzOH = benzyl alcohol;
EtOH = ethanol;
WFI = water for injection

The resulting formulations were administered by subcutaneous injection to Sprauge-Dawley rats (n=6 per group) according to Table 2. Blood samples, collected by sub-lingual bleeding, for pharmacokinetic analysis were drawn pre-dose, and then 1 hour, 6 hours, 1 day, 2 days, 5 days, and 8 days after dosing. The blood was placed on ice immediately after collection and centrifuged (1,200×g, 2-5° C., and 10 min) within 30 to 60 minutes. The plasma was transferred into 1.5-mL propylene test tubes (Microcentrifuge tubes, Plastibrand, Buch & Holm) and stored below −70° C. until analysis. The concentration of GRN in the rat plasma was analysed using HPLC, a reversed phase gradient HPLC method with UV-detection. The plasma samples were purified on solid phase extraction (SPE) columns prior to HPLC analysis.

TABLE 2
Dosing of pre-formulations comprising GRN and GRN (Cl)

| | | | | Dose | Dose |
|---|---|---|---|---|---|
| Group | No of | Treatment | Route of | of GRN* | volume |
| No | animals | (Formulation) | administration | (mg/kg) | (mL/kg) |
| 1 | 6 | 1A | s.c. | 40 | 1.0 |
| 2 | 6 | 1A | s.c. | 13.2 | 0.33 |
| 3 | 6 | 1B | s.c. | 35.8 | 1.0 |
| 4 | 6 | 1B | s.c. | 11.8 | 0.33 |

*Calculated as granisetron free base

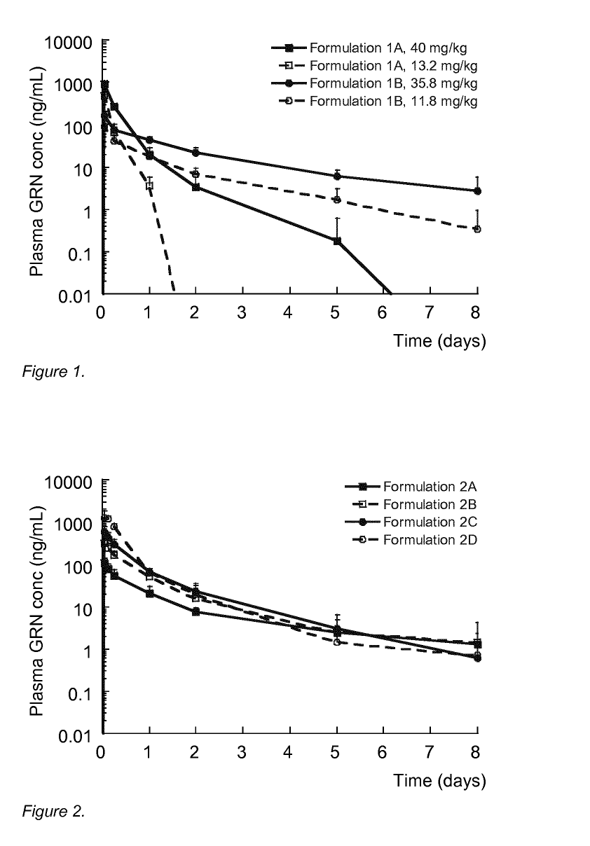

The results are shown in FIG. 1. Comparison of Formulation 1A with Formulation 1B shows that Formulation 1A, i.e., containing no polar solvent, had a markedly higher initial release rate of granisetron. In addition, Formulation 1B produced a much longer release duration, there being a detectable level of granisetron after 8 days.

### Example 2

Pre-formulations (5 g) were produced according to the procedure outlined in Example 1 using granisetron chloride (GRN(Cl)) as the API with water as the polar solvent, having the compositions shown in Table 3.

TABLE 3
Formulation compositions (wt %).

| Formulation | GRN (Cl) | GDO | SPC | EtOH | WFI |
|---|---|---|---|---|---|
| 2A | 2.00 | 31.50 | 31.50 | 15.00 | 20.00 |
| 2B | 4.00 | 30.50 | 30.50 | 15.00 | 20.00 |
| 2C | 6.00 | 29.50 | 29.50 | 15.00 | 20.00 |
| 2D | 9.00 | 28.00 | 28.00 | 15.00 | 20.00 |

Abbreviations:
GRN (Cl) = granisetron hydrochloride;
GDO = glycerol dioleate;
SPC = soy phosphatidylcholine;
EtOH = ethanol;
WFI = water for injection

Following the procedure described for Example 1, the release duration of granisetron after subcutaneous injection was evaluated in rats using the doses set out in Table 4.

TABLE 4
Dosing of pre-formulations comprising GRN (Cl)

| | | | | Dose of | Dose |
|---|---|---|---|---|---|
| Group | No of | Treatment | Route of | GRN (Cl) | volume |
| No | animals | (Formulation) | administration | (mg/kg) | (mL/kg) |
| 1 | 6 | 2A | s.c. | 20.0 | 1.0 |
| 2 | 6 | 2B | s.c. | 40.0 | 1.0 |
| 3 | 6 | 2C | s.c. | 60.0 | 1.0 |
| 4 | 6 | 2D | s.c. | 90.0 | 1.0 |

The results are shown in FIG. 2. All formulations produced granisetron release duration of at least 8 days. A somewhat faster initial release was observed for Formulation 2D (9 wt % GRN(Cl) corresponding to ca 90 mg/mL).

### Example 3

Pre-formulations comprising granisetron hydrochloride (GRN(Cl)) were prepared according to the procedure outlined in Example 1, having the compositions shown in Table 5. The formulations had 3.0, 3.5 or 4.0 wt % of GRN(Cl) corresponding to ca 30, 35 or 40 mg/mL of the active ingredient. The formulations were clear and homogenous after mixing and remained as such after long-term storage at room temperature (>1 month). Viscosity measurements of formulations 3A-3D were performed using CAP 2000+ high torque viscometer (Brookfield, Mass.) equipped with CAP01 cone spindle at a share rate rotation speed 300 rpm (500 rpm for 3E formulation) at 20 and 25° C. 75 μl of the formulation was placed between holding plate and cone spindle, equilibrated for 10 s and measured for 15 s. The viscosity results are included in Table 5.

TABLE 5
Formulation compositions (wt %) and viscosities.

| | | | | | | Viscosity (cP) | |
|---|---|---|---|---|---|---|---|
| Formulation | GRN(Cl) | SPC | GDO | EtOH | WFI | 20° C. | 25° C. |
| 3A | 3.0 | 38.5 | 38.5 | 10.0 | 10.0 | 286.6 | 229.1 |
| 3B | 3.5 | 37.25 | 37.25 | 12.0 | 10.0 | 200.0 | 162.6 |
| 3C | 4.0 | 35.5 | 35.5 | 15.0 | 10.0 | 122.8 | 97.2 |
| 3D | 4.0 | 33.0 | 33.0 | 15.0 | 15.0 | 153.2 | 118.8 |

Abbreviations:
GRN(Cl) = granisetron hydrochloride;
SPC = soy phosphatidylcholine;
GDO = glycerol dioleate;
EtOH = ethanol;
WFI = water for injection

### Example 4

Pre-formulations comprising granisetron hydrochloride (GRN(Cl)) were prepared by first mixing lipids and solvents to form homogenous solutions followed by addition of GRN(Cl) powder and continuous mixing by end-over-end rotation at ambient RT until completely clear and homogenous formulations were achieved. The final compositions are shown in Table 6. The formulations were all clear and homogenous liquids and had between 0.5-4.0 wt % of GRN(Cl) corresponding to ca 5-40 mg/mL of the active ingredient.

TABLE 6
Formulation compositions (wt %) and SPC/GDO weight ratios.

| | | | | | | SPC/GDO |
|---|---|---|---|---|---|---|
| Formulation | GRN(Cl) | SPC | GDO | EtOH | WFI | weight ratio |
| 4A | 1.0 | 39.5 | 39.5 | 10 | 10 | 50/50 |
| 4B | 2.0 | 39.0 | 39.0 | 10 | 10 | 50/50 |
| 4C | 3.0 | 38.5 | 38.5 | 10 | 10 | 50/50 |
| 4D | 0.5 | 37.3 | 37.3 | 15 | 10 | 50/50 |
| 4E | 1.0 | 37.0 | 37.0 | 15 | 10 | 50/50 |
| 4F | 1.5 | 36.8 | 36.8 | 15 | 10 | 50/50 |
| 4G | 2.0 | 36.5 | 36.5 | 15 | 10 | 50/50 |
| 4H | 2.5 | 36.3 | 36.3 | 15 | 10 | 50/50 |
| 4I | 3.0 | 36.0 | 36.0 | 15 | 10 | 50/50 |
| 4J | 3.4 | 35.8 | 35.8 | 15 | 10 | 50/50 |
| 4K | 4.0 | 35.5 | 35.5 | 15 | 10 | 50/50 |
| 4L | 1.0 | 34.5 | 34.5 | 15 | 15 | 50/50 |
| 4M | 2.0 | 34.0 | 34.0 | 15 | 15 | 50/50 |
| 4N | 3.0 | 33.5 | 33.5 | 15 | 15 | 50/50 |
| 4O | 4.0 | 33 | 33 | 15 | 15 | 50/50 |
| 4P | 1.0 | 38.5 | 38.5 | 12 | 10 | 50/50 |
| 4Q | 2.0 | 38.0 | 38.0 | 12 | 10 | 50/50 |
| 4R | 3.0 | 37.5 | 37.5 | 12 | 10 | 50/50 |
| 4S | 3.5 | 37.3 | 37.3 | 12 | 10 | 50/50 |
| 4T | 0.5 | 26.1 | 48.4 | 15 | 10 | 35/65 |
| 4U | 1.0 | 25.9 | 48.1 | 15 | 10 | 35/65 |
| 4V | 1.5 | 25.7 | 47.8 | 15 | 10 | 35/65 |
| 4X | 2.0 | 25.5 | 47.5 | 15 | 10 | 35/65 |
| 4Y | 2.5 | 25.4 | 47.1 | 15 | 10 | 35/65 |
| 4Z | 3.0 | 25.2 | 46.8 | 15 | 10 | 35/65 |
| 4AA | 3.4 | 25.1 | 46.5 | 15 | 10 | 35/65 |
| 4AB | 4.0 | 24.9 | 46.1 | 15 | 10 | 35/65 |

Abbreviations:
GRN(Cl) = granisetron hydrochloride;
SPC = soy phosphatidylcholine;
GDO = glycerol dioleate;
EtOH = ethanol;
WFI = water for injection

About 100 mg of the respective formulation in Table 6 was injected into 5 mL phosphate buffered saline (PBS, pH 7.4) and left to equilibrate at ambient temperature in still standing vials for 8 days before Small Angle X-ray Diffraction (SAXD) measurements. The nanostructure of the fully hydrated lipid samples was studied using synchrotron SAXD measurements, performed at the I911-4 beamline at MAX IV laboratory (Max II electron accelerator operating at 1.5 GeV, Lund University, Sweden), using a 1M PILATUS 2D detector (Dectris) containing a total of 981×1043 pixels.

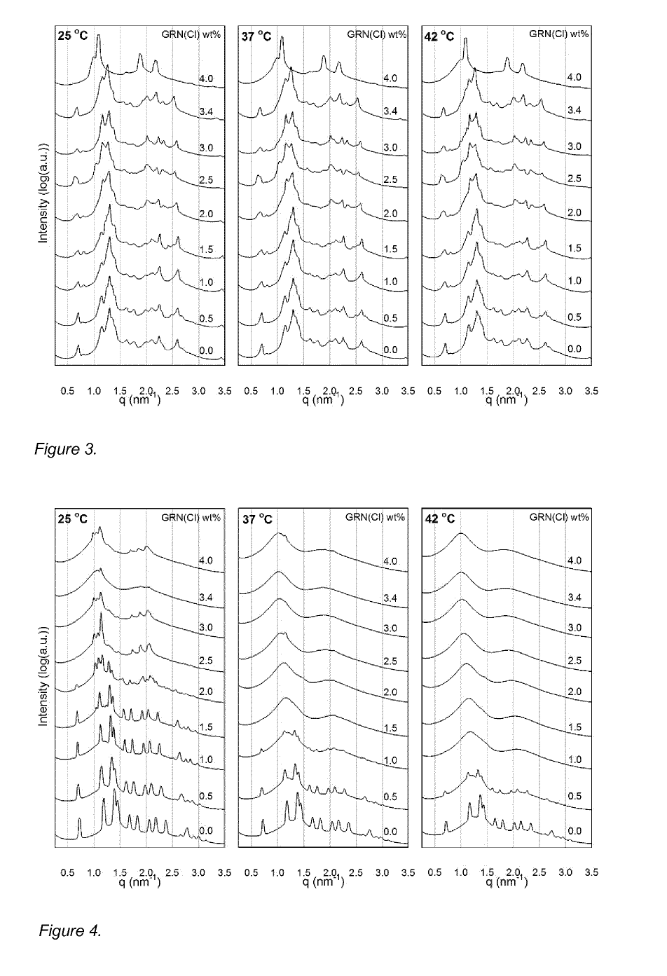

FIG. 3 shows obtained SAXD results of the nanostructure of the fully hydrated Lipid/EtOH/WFI mixtures as a function of GRN(Cl) concentration and temperature. The SPC/GDO weight ratio was fixed to 50/50 and the EtOH/WFI to 15/10. Data show that independent of temperature, all fully hydrated formulations formed mixtures of reversed hexagonal and reversed micellar cubic Fd3m phases up to 3.4 wt % of GRN(Cl). With increasing GRN(Cl) concentration the lattice parameter for the Fd3m phase remained unchanged whereas it slightly increased for the hexagonal phase. At 4 wt % of GRN(Cl), a mixture of a more swollen hexagonal phase and a phase which is different from Fd3m is formed. Notably, the liquid crystal structure is stable at all GRN(Cl) loading levels at and above physiological temperature (37-42° C.).

Similar results were obtained for samples with the SPC/GDO weight ratio fixed at 50/50 and with EtOH/WFI weight ratios at 12/10 and 10/10.

In contrast to the temperature stable liquid crystal (LC) nanostructures formed with the SPC/GDO weight ratio of 50/50, the effect of GRN(Cl) on the liquid crystalline nanostructure on samples prepared at a SPC/GDO weight ratio of 35/65 was much more pronounced (FIG. 4). At 25° C. and up to 1.0 wt % of GRN(Cl), a single Fd3m phase is formed; at 1.5 wt % of GRN(Cl), a 3D hexagonal (P63/mmc symmetry) phase starts to appear which in single form is found starting from 2.5 wt % GRN(Cl); at 3.4 and 4.0 wt % of GRN(Cl), the 3D hexagonal P63/mmc phase is almost completely lost and transformed into a disordered solution of reversed micelles, L2. This is clearly shown by the broad and featureless diffraction peaks. Notably, the L2 phase, or in other words, a disordered arrangement of the reversed micelles starts to emerge already at 1 wt % of GRN(Cl) (visible from the appearance of broad diffraction peaks) and coexists together with liquid crystalline phases all the way as GRN(Cl) concentration is increased. At elevated temperatures (37 and 42° C.), the Fd3m liquid crystalline phase can accommodate only 0.5-1.0 wt % GRN(Cl) before its full transformation to L2. Hence, the SPC/GDO 35/65 wt/wt composition is not able to accommodate more than 0.5-1.0 wt % of GRN(Cl) at, and slightly above, physiological temperature before a complete transformation of the liquid crystal structure. This effect significantly restricts the usefulness of such compositions for long-acting release of granisetron.

### Example 5

Pre-formulations comprising a combination of granisetron hydrochloride (GRN(Cl)) and buprenorphine (BUP) were prepared according to the procedure outlined in Example 1, having the compositions shown in Table 7. The formulations were clear and homogenous after mixing and remained as such after long-term storage in the temperature interval 15-25° C. (>1 month). The formulations had 2 wt % of GRN(Cl) and between 0.4-1.6 wt % BUP corresponding to ca 20 mg/mL GRN(Cl) and ca 4-16 mg/mL BUP.

TABLE 7
Formulation compositions comprising
granisetron and buprenorphine (wt %).

| Formulation | GRN(Cl) | BUP | SPC | GDO | EtOH | WFI |
|---|---|---|---|---|---|---|
| 5A | 2.0 | 2.0 | 33.0 | 33.0 | 15.0 | 15.0 |
| 5B | 2.0 | 2.0 | 35.5 | 35.5 | 15.0 | 10.0 |
| 5C | 2.0 | 1.6 | 37.2 | 37.2 | 12.0 | 10.0 |
| 5D | 2.0 | 0.4 | 37.8 | 37.8 | 12.0 | 10.0 |
| 5E | 2.0 | 2.0 | 37.0 | 37.0 | 12.0 | 10.0 |
| 5F | 2.0 | 2.0 | 38.0 | 38.0 | 10.0 | 10.0 |
| 5G | 2.0 | 1.6 | 38.2 | 38.2 | 10.0 | 10.0 |
| 5H | 2.0 | 0.4 | 38.8 | 38.8 | 10.0 | 10.0 |

Abbreviations:
GRN(Cl) = granisetron hydrochloride;
BUP = buprenorphine;
SPC = soy phosphatidylcholine;
GDO = glycerol dioleate;
EtOH = ethanol;
WFI = water for injection

### Example 6

Pre-formulations comprising ondansetron hydrochloride (ONN(Cl)) are prepared according to the procedure outlined in Example 1, having the compositions shown in Table 8. The formulations have between 2-4 wt % of ONN(Cl) corresponding to ca 20-40 mg/mL ONN(Cl).

TABLE 8
Formulation compositions comprising ondansetron (wt %).

| Formulation | ONN (Cl) | SPC | GDO | EtOH | WFI |
|---|---|---|---|---|---|
| 6A | 2.0 | 35.5 | 35.5 | 15.0 | 10.0 |
| 6B | 4.0 | 33.0 | 33.0 | 15.0 | 15.0 |

Abbreviations:
ONN (Cl) = ondansetron hydrochloride;
SPC = soy phosphatidylcholine;
GDO = glycerol dioleate;
EtOH = ethanol;
WFI = water for injection

### Example 7

Pre-formulations (ca 180 g) comprising either granisetron hydrochloride (GRN(Cl)) or a combination of GRN(Cl) and buprenorphine (BUP) were prepared according to the procedure outlined in Example 1, having the compositions shown in Table 9.

TABLE 9
Formulation compositions (wt %) comprising GRN(Cl) and BUP.

| | | | | | | CA buffer | |
|---|---|---|---|---|---|---|---|
| | | | | | | (20 mM) | |
| Formulation | GRN(Cl)¹ | BUP² | SPC | GDO | EtOH | pH 4.5³ | WFI |
| 7A | 2.34 | — | 37.83 | 37.83 | 12.00 | 10.00 | — |
| 7B | 2.34 | 1.30 | 37.18 | 37.18 | 12.00 | — | 10.00 |

¹The GRN(Cl) wt % corresponds to a concentration of 20.0 mg/mL GRN free base, after correction for chloride content density of the formulation.
²The BUP wt % corresponds to a concentration of 12.5 mg/mL BUP free base, after correction for the density of the formulation.
³Citric acid (CA) buffer (20 mM), pH 4.5

The in vivo release profiles of granisetron and buprenorphine after subcutaneous injection was evaluated in rats using the doses set out in Table 10. Quantification of GRN in rat plasma was performed as outlined in Example 1 whereas the BUP concentration in EDTA rat plasma samples was analysed by ELISA.

TABLE 10
Dosing of pre-formulations comprising
GRN(Cl) or GRN(Cl) and BUP.

| | | Test Item | Route of | Dose | Dose of | Dose of |
|---|---|---|---|---|---|---|
| Group | No of | (Formu- | adminis- | volume | GRN | BUP |
| no | animals | lation) | tration | (mL/kg) | (mg/kg) | (mg/kg) |
| 1 | 6 | Formu- | s.c. | 0.67 | 13.4 | — |
| | | lation 7A | | | | |
| 2 | 6 | Formu- | s.c. | 0.67 | 13.4 | 8.38 |
| | | lation 7B | | | | |

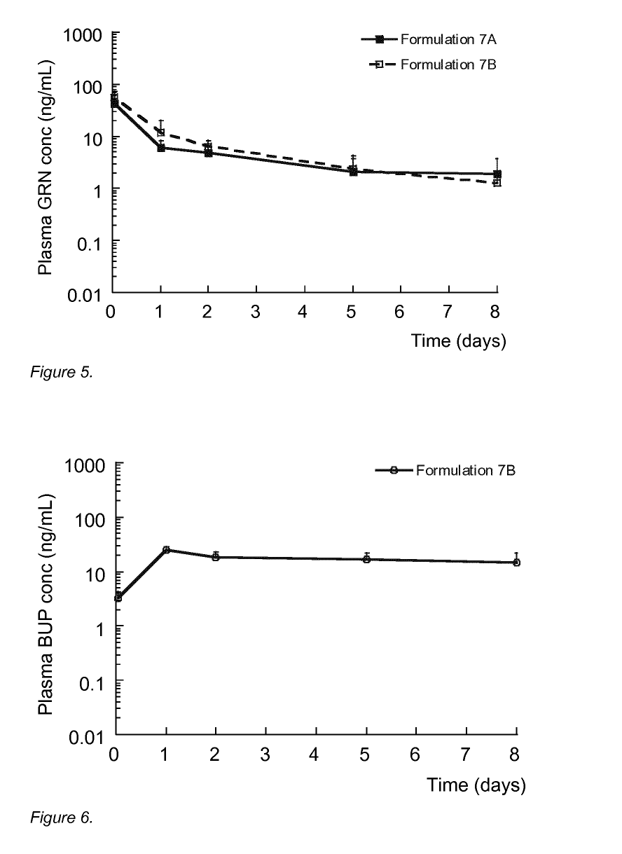

The results are shown in FIG. 5 and FIG. 6. Both formulations produced granisetron release duration of at least 8 days with similar pharmacokinetic (PK) profiles (FIG. 5). Formulation 7B in addition provided consistent buprenorphine release over at least 8 days (FIG. 6).

### Example 8

A pre-formulation (150 g) comprising a combination of granisetron hydrochloride (GRN(Cl)) and buprenorphine (BUP) and including the antioxidant mono-thioglycerol (MTG) was prepared according to the procedure outlined in Example 1, having the composition shown in Table 11.

TABLE 11
Formulation composition (wt %) comprising
GRN(Cl), BUP and antioxidant MTG.

| Formulation | GRN(Cl)¹ | BUP² | SPC | GDO | EtOH | WFI | MTG³ |
|---|---|---|---|---|---|---|---|
| 8A | 2.34 | 1.30 | 36.93 | 36.93 | 12.00 | 10.00 | 0.50 |

¹The GRN(Cl) wt % corresponds to a concentration of 20.0 mg/mL GRN free base, after correction for chloride content and density of the formulation.
²The BUP wt % corresponds to a concentration of 12.5 mg/mL BUP free base, after correction for the density of the formulation.
³Mono-thioglycerol

### Example 9

A pre-formulation (150 g) comprising a combination of granisetron hydrochloride (GRN(Cl)) and buprenorphine (BUP) was prepared according to the procedure outlined in Example 1, having the composition shown in Table 12.

TABLE 12
Formulation composition (wt %) comprising GRN(Cl) and BUP.

| Formulation | GRN(Cl)¹ | BUP² | SPC | GDO | EtOH | WFI |
|---|---|---|---|---|---|---|
| 8A | 2.34 | 0.65 | 37.50 | 37.50 | 12.00 | 10.00 |

¹The GRN(Cl) wt % corresponds to a concentration of 20.0 mg/mL GRN free base, after correction for chloride content and density of the formulation.
²The BUP wt % corresponds to a concentration of 6.25 mg/mL BUP free base, after correction for the density of the formulation.

### Example 10

The in vivo release profiles of granisetron and buprenorphine after subcutaneous injection of Formulations 7A and 7B (Example 7) in dog (beagle) was evaluated using the doses set out in Table 13. The animals were dosed on Days 1 and 8 with the respective formulation.

TABLE 13
Dosing of pre-formulations comprising
GRN(Cl) or GRN(Cl) and BUP.

| | | Test Item | Route of | Dose | Dose of | Dose of |
|---|---|---|---|---|---|---|
| Group | No of | (Formu- | adminis- | volume | GRN | BUP |
| no | animals | lation) | tration | (mL) | (mg) | (mg) |
| 1 | 6 | Formu- | s.c. | 0.64 | 12.8 | — |
| | | lation 7A | | | | |
| 2 | 6 | Formu- | s.c. | 1.28 | 25.6 | — |
| | | lation 7A | | | | |
| 3 | 6 | Formu- | s.c. | 0.64 | 12.8 | 8.0 |
| | | lation 7B | | | | |
| 4 | 6 | Formu- | s.c. | 1.28 | 25.6 | 16.0 |
| | | lation 7B | | | | |

A validated bioanalytical method was used for the determination of BUP in dog plasma. The analytical method employed liquid chromatography with tandem mass spectrometric detection. The lower limit of quantification of the analytical method was 0.100 ng/mL. The calibration curve ranged from 0.100 to 50.0 ng/mL. Samples above the upper limit of quantitation were diluted into range using control beagle K2-EDTA plasma.

A validated bioanalytical method was used for the determination of GRN in dog plasma. The analytical method employed liquid chromatography with tandem mass spectrometric detection. The lower limit of quantification of the analytical method was 0.05 ng/mL. The calibration curve ranged from 0.05 to 50.0 ng/mL. Samples above the upper limit of quantitation were diluted into range using control beagle K2-EDTA plasma.

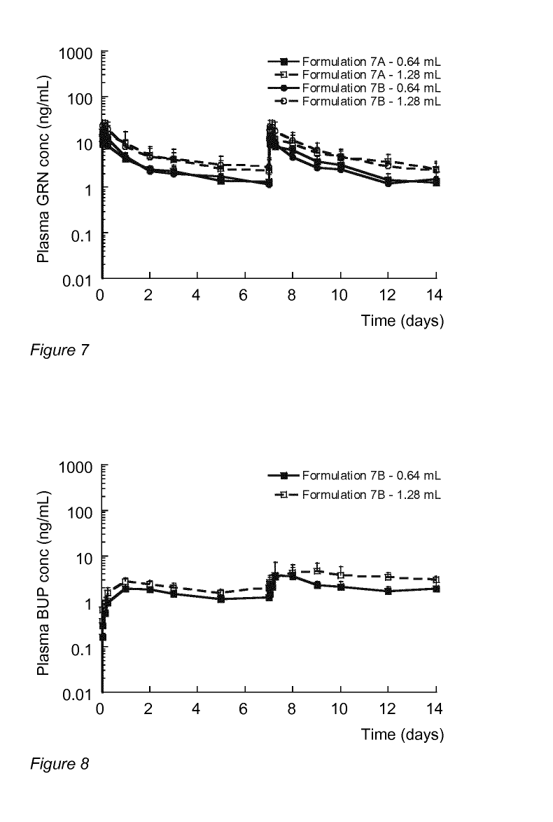

The results are shown in FIG. 7 and FIG. 8. Both formulations produced granisetron release duration of at least 7 days with similar pharmacokinetic (PK) profiles (FIG. 7). Formulation 7B also provided consistent buprenorphine release over at least 7 days (FIG. 8). In addition, dose linearity was indicated for both granisetron and buprenorphine with respect to exposure (AUC) over the 2 weekly dose intervals.

1. An injectable pre-formulation comprising a low viscosity mixture of:
a) at least one neutral diacyl lipid and/or at least one tocopherol;
b) at least one phospholipid;
c) at least one oxygen containing organic solvent;
d) at least one 5HT₃ antagonist;
wherein the pre-formulation forms, or is capable of forming, at least one liquid crystalline phase structure upon contact with an aqueous fluid.

2. A pre-formulation as claimed in claim 1 wherein component a) comprises at least one diacyl glycerol.

3. A pre-formulation as claimed in either of claim 1 or 2 wherein component a) comprises, consists essentially of or consists of glycerol dioleate.

4. A pre-formulation as claimed in any of claims 1 to 3 further comprising a polar solvent component e).

5. A pre-formulation as claimed in any of claims 1 to 4 wherein component d) comprises at least one 5HT₃ antagonist or second generation 5HT₃ agonist, preferably selected from ondansetron, tropisetron, granisetron, dolasetron, palonosetron, alosetron, cilansetron and/or ramosetron or mixtures thereof.

6. A pre-formulation as claimed in claim 5 wherein component d) comprises or consists of granisetron or a biologically acceptable salt thereof.

7. A pre-formulation as claimed in claim 6 wherein component d) comprises or consists of granisetron chloride.

8. A pre-formulation as claimed in any of claims 1 to 7 comprising 0.1-25 wt. % granisetron (calculated as granisetron free base), preferably 0.5-15 wt. %, most preferably 1-15 wt. %, especially 1-10 wt. %, such as 1.6-10 wt. %.

9. A pre-formulation as claimed in any of claims 1 to 8 wherein component a) is present in an amount of 20-54 wt. % of the pre-formulation, preferably 20-45 wt. %, most preferably 25-40 wt. %.

10. A pre-formulation as claimed in any of claims 1 to 9 wherein component a) comprises or consists of GDO.

11. A pre-formulation as claimed in any of claims 1 to 10 wherein component b) is present in an amount of 20-60 wt. % of the pre-formulation, preferably 25-50 wt. %, most preferably 25-45 wt. %.

12. A pre-formulation as claimed in any of claims 1 to 11 wherein the ratio of components a):b) (w/w) is in the range of 40:60 to 64:36, preferably in the range of 45:55 to 55:45.

13. A pre-formulation as claimed in any of claims 1 to 12 wherein component c) comprises or consists of a phosphatidylcholine, preferably soy PC.

14. A pre-formulation as claimed in any of claims 1 to 13 wherein component c) is present in an amount of 1-30 wt. % of the pre-formulation, preferably 5-20 wt. %, more preferably 7-18 wt. %.

15. A pre-formulation as claimed in any of claims 1 to 14 wherein component c) comprises or consists of ethanol, benzyl alcohol, NMP, DMSO, or mixtures thereof.

16. A pre-formulation as claimed in any of claims 1 to 15 wherein component e) is present in an amount of 5-35 wt. % of the pre-formulation, preferably 8-30 wt. %, most preferably 10-30 wt. %.

17. A pre-formulation as claimed in any of claims 1 to 16 wherein the polar solvent component e) comprises or consists of water, propylene glycol, or mixtures of water and propylene glycol.

18. A pre-formulation as claimed in any of claims 1 to 17 further comprising f) at least one opioid agonist, partial agonist and/or antagonist.

19. A pre-formulation as claimed in claim 18 wherein component f) comprises or consists of buprenorphine or a biologically acceptable salt thereof.

20. A pre-formulation claimed in any of claims 1 to 19 wherein component d) comprises or consists of granisetron or a biologically acceptable salt thereof, and wherein component f) comprises or consists of buprenorphine or a biologically acceptable salt thereof.

21. A pre-formulation as claimed in any of claims 1 to 20 wherein the weight ratio of opioid (free base):5HT3 antagonist (free base) (d):(f) is in the range of 5:95 to 95:5, preferably in the range of 15:85 to 85:15 or 25:75 to 75:25.

22. A pre-formulation as claimed in any of claims 1 to 21 wherein component f) further comprises naloxone or a biologically acceptable salt thereof.

23. A pre-formulation as claimed in claim 22 wherein the ratio of naloxone:opioid agonist and/or antagonist is 1:7 to 1:2, preferably 1:6 to 1:3.

24. A pre-formulation as claimed in claim 23 wherein the opioid is buprenorphine.

25. A pre-formulation as claimed in any of claims 1 to 24 administrable through a needle no larger than 19 gauge, preferably 23 gauge, under manual pressure.

26. A pre-formulation as claimed in any of claims 1 to 25 having a viscosity of 1 to 1000 mPas at 20° C., preferably 100 to 700 mPas at 20° C.

27. A pre-formulation according to any preceding claim that is suitable for parenteral administration.

28. A depot product formed by contacting a pre-formulation as claimed in any of claims 1 to 27 with an aqueous fluid.

29. A method for the treatment of a human or non-human mammalian subject in need thereof with a 5HT3 antagonist, said method comprising administering to said subject an injectable pre-formulation comprising a low-viscosity mixture of;
a) at least one neutral diacyl lipid;
b) at least one phospholipid;
c) at least one oxygen containing organic solvent; and
d) at least one 5HT3 antagonist.

30. The method as claimed in claim 29 wherein the method of treatment is a method for the treatment of at least one condition selected from emesis, pain, nausea, chemotherapy and/or radiotherapy and/or endoradionucleotide induced nausea and vomiting, post-operative nausea and vomiting, opioid dependence, cancer, motion sickness, IBS, and/or related conditions.

31. The method as claimed in either of claim 29 or 30 comprising the administration of at least one pre-formulation as claimed in any of claims 1 to 27.

32. The method as claimed in any of claims 29 to 31 comprising administration by spray, i.m., s.c. or deep s.c. injection.

33. The method as claimed in any of claims 29 to 32 comprising administration by means of a pre-filled administration device.

34. The method as claimed in any of claims 29 to 33 comprising administration through a needle no larger than 19 gauge, preferably 23 gauge.

35. The method as claimed in any of claims 29 to 34 comprising a single administration every 1 to 14 days, preferably every 1 to 7 days, more preferably every 3 to 7 days.

36. The method as claimed in any of claims 29 to 35 where the administered dose is selected at the point of delivery.

37. The method of claim 36 where the selected dose is chosen relative to the weight of the subject.

38. The method of claim 36 or 37 where the administered dose is selected by means of the volume injected.

39. Use of:
a) at least one neutral diacyl lipid;
b) at least one phospholipid;
c) at least one oxygen containing organic solvent; and
d) at least one 5HT3 antagonist;
in the manufacture of an injectable low viscosity pre-formulation medicament for use in the in vivo formation of a depot for treatment of at least one condition selected from emesis, pain, nausea, chemotherapy and/or radiotherapy and/or endoradionucleotide induced acute or delayed nausea and vomiting, post-operative nausea and vomiting, opioid dependence, cancer, motion sickness, IBS and/or related conditions.

40. The use as claimed in claim 39 comprising the use of at least one formulation as claimed in any of claims 1 to 27.

41. An injectable pre-formulation comprising a low viscosity mixture of:
a) at least one neutral diacyl lipid;
b) at least one phospholipid;
c) at least one oxygen containing organic solvent;
d) at least one 5HT₃ antagonist;
wherein the pre-formulation forms, or is capable of forming, at least one liquid crystalline phase structure upon contact with an aqueous fluid;
for use in the treatment of at least one condition selected from emesis, pain, nausea, chemotherapy and/or radiotherapy and/or endoradionucleotide induced acute or delayed nausea and vomiting, post-operative nausea and vomiting, opioid dependence, cancer, motion sickness, IBS and/or related conditions.

42. An injectable pre-formulation for use as claimed in claim 41, wherein the pre-formulation is as claimed in any of claims 1 to 27.

43. A disposable administration device pre-loaded with a measured dose of a preformulation comprising an injectable low viscosity mixture of:
a) at least one neutral diacyl lipid;
b) at least one phospholipid;
c) at least one oxygen containing organic solvent; and
d) at least one 5HT3 antagonist.

44. The device of claim 43 being a syringe and syringe barrel.

45. The device of claim 43 or 44 containing a formulation as claimed in claims 1 to 27.

46. The device of any of claims 43 to 45 comprising a needle no larger than 19 gauge, preferably no larger than 23 gauge.

47. The device of any of claims 43 to 46 containing granisetron at around 0.2 to 3 mg per day between scheduled administrations.

48. The device of any of claims 43 to 47 containing a total volume for administration of no more than 5 ml.

49. A kit for the administration of at least one 5HT3 antagonist, said kit containing a measured dose of a formulation comprising an injectable low viscosity mixture of:
a) at least one neutral diacyl lipid;
b) at least one phospholipid;
c) at least one oxygen containing organic solvent; and
d) at least one 5HT3 antagonist.

50. The kit of claim 49 including an administration device.

51. The kit of claim 49 or 50 containing a preferred formulation as indicated herein.

52. The kit of any of claims 49 to 51 containing a prefilled device as indicated in any of claims 43 to 48.

53. The kit of any of claims 49 to 52 containing a needle no larger than 19 gauge, preferably no larger than 23 gauge.

54. The kit of any of claims 49 to 53 containing granisetron at around 0.2 to 3 mg per day between scheduled administrations.

55. The kit of any of claims 49 to 54 containing a total volume for administration of no more than 5 ml, preferably no more than 3 ml more preferably no more than 2 ml.

56. The kit of any of claims 49 to 55 containing instructions for administration by spray, i.m, s./c or deep s.c. injection.

57. The kit of any of claims 49 to 56 containing instructions for administration for use in a method of treatment as described any of claims 29 to 38.
